(19) United States
(12) Patent Application Publication
(10) Pub. No.: US 2008/0275545 A1
(43) Pub. Date: Nov. 6, 2008

(54) Method and Device for Investigation of Sludge Deposits on Materials for Endoprostheses and Endoprosthesis

(76) Inventors: Uwe Seitz, Hamburg (DE); Uwe Wienhold, Magdeburg (DE); Carsten Grosse, Daldorf (DE)

Correspondence Address: BRINKS HOFER GILSON & LIONE/CHICAGO/COOK, PO BOX 10395, CHICAGO, IL, 60610, US

(21) Appl. No.: 11/596,751

(22) PCT Filed: May 17, 2005
(86) PCT No.: PCT/DE2005/000912

§ 371 Date: Apr. 25, 2008

(30) Foreign Application Priority Data
May 18, 2004 (DE) 102004025130.4

(51) Int. Cl. A61F 2/04; G01N 17/00; G01N 33/48; G01N 35/00; C12M 3/00

(52) U.S. Cl. 623/1.46; 435/303.3; 436/5; 436/6; 623/1.49

(57) ABSTRACT

The invention relates to a method and device for the investigation of sludge deposition on materials for coating endoprostheses. At least one endoprosthesis is placed in an incubation chamber which is at least partly filled with infected bile. The incubation chamber is rocked gently on a rocking platform for a period of about 35 days. The bile fluid is regularly partly exchanged. The endoprosthesis is embodied in a coated plastic. The coating comprises 70 to 80% by weight of hydrophobic components and 30% to 20% by weight of hydrophilic components. The coating may particularly have a sol-gel embodiment. An application in bile duct prostheses is possible.

The invention relates to a method for the investigation of sludge deposits on materials for the coating of endoprostheses.

Moreover, the invention relates to a device for the investigation of materials for the coating of endoprostheses.

The invention also relates to an endoprosthesis made of a plastic with a coating.

Patients with advanced cancer of the head of the pancreas have a low life expectancy and, due to the stenosis of the extrahepatic bile duct, often suffer from cholestasis with resulting massive itching and the risk of secondary cholangitis.

The method of choice for the palliative therapy for these patients is the endoscopic stenting of the bile duct with plastic prostheses, in order to ensure bile drainage.

The problem of the obstruction of the prostheses by so-called “sludge” made up of bacteria, calcium bilirubinate, etc. after a lying duration of approx. 3-6 months could not be solved up to the present time even through different modifications (e.g. different plastics or coatings, the addition of antibiotics and construction modifications). Obstructed stents must be replaced with new ones.

Metal stents used as an alternative are only rarely used in clinics due to approx. 20 times higher materials costs.

Adenocarcinoma of the pancreas represents the fourth most common (in men) or fifth most common (in women) malignant tumor and has the lowest five-year survival rate of all types of cancers (1-5%). The incidence of pancreatic tumors has been increasing for years in the important industrial states and has currently reached 8-13 cases per 100,000 people.

The risk of contraction increases considerably after the age of fifty. Diagnosis is most often made in the age range from 65 to 80.

The extremely poor five-year survival rate is caused by the failure to detect the disease early on, then, at the time of diagnosis, most patients already have an advanced tumor and distant metastases (52%). Thus, the surgical resection can only be performed from a curative point of view in 14% of patients.

As proven in distant metastases and extensive vascular infiltration of the portal vein system, a curative therapy concept is impossible for the Arteria mesenerica superior and the Arteria hepatica.

Thus, only a palliative therapy concept can be considered for these patients, who form the overwhelming majority in everyday clinical practice.

Depending on patients' symptoms/complaints, the endoscopic insertion of stents into the bile duct and the duodenum as well as surgical measures (gastroenterostomy, biliodigestive anastomosis) are available in addition to pain therapy and palliative chemotherapy.

In comparison with palliative surgical measures, endoscopic methods have a lower rate of morbidity, a lower mortality within 30 days and are less expensive. At the same time, the patient experiences a higher quality of life.

Thus, endoscopic inventions are increasingly favored, unless, at the time of diagnosis, there is already a significant stomach outlet stenosis due to tumor infiltration.

The first choice method for tumor-related icterus is the implantation of a biliary endoprosthesis and was already introduced in 1979.

Today, this occurs by means of endoscopic retrograde cholangiopancreatography (ERCP) and the vast experience of the operator in over 95% of patients and is associated with a reduction or a normalization of the extrahepatic cholestasis and thus of the cholangitis risk in almost all patients.

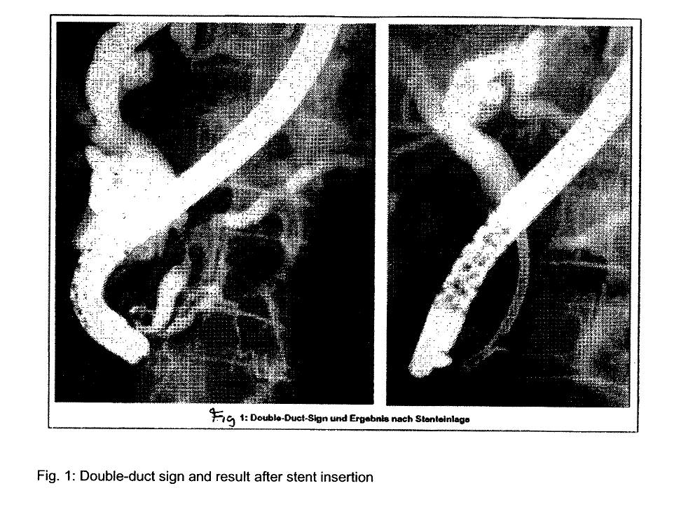

FIG. 1 shows the typical “Double Duct Sign” in a blocked pancreas and bile duct for an inoperable tumor in the head of the pancreas as well as the result after endoscopic insertion of a bile duct endoprostheses.

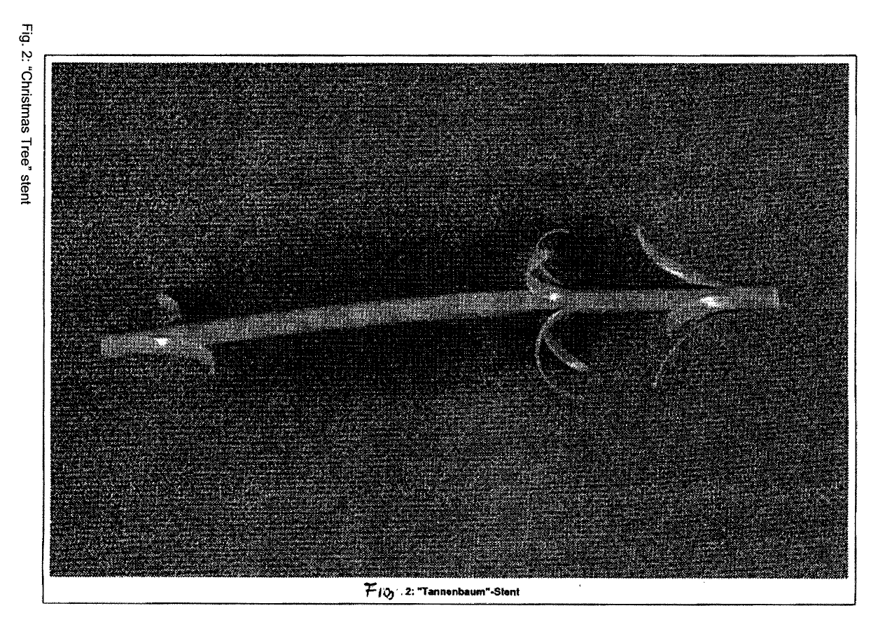

The main prostheses for transpapillary bile drainage currently used in the Clinic for Interdisciplinary Endoscopy in the UKE are so-called “Christmas tree stents” made of Teflon shown in FIG. 2. The preferred caliber for a permanently good functioning of the prosthesis is thereby 10-12 French (1 French=⅓ mm).

A known and up to now not definitively solved problem is the blocking of the prostheses after a few months of lying time.

In rare cases, dislocations in the surrounding area have also caused insufficient bile drainage.

The patients then require a stent replacement due to the returning icterus with increasing cholestasis parameters and the risk of a secondary cholangitis. Since the introduction of the “stent retriever,” this has been technically easy to implement, but also means an ambulant reassessment for the re-intervention and a restriction in the patient's quality of life.

After insertion, the proteins like fibronectin, collagen, fibrin and immunoglobulin adhere to the surface of the stent material. These proteins promote the adhesion of bacteria and the formation of a “biofilm,” consisting of bacteria and bacterially developed glycoproteins.

The bacterial enzyme β glucuronidase, which is above all produced from Escherichia coli, deconjugates bilirubin and precipitates it as salt.

A sludgy-sandy, yellow-orange to black-brown colored material, which is called “sludge,” is created from the accumulation of bacteria, glycoproteins, plant fibers and crystals of calcium bilirubinate and calcium fatty acid salts.

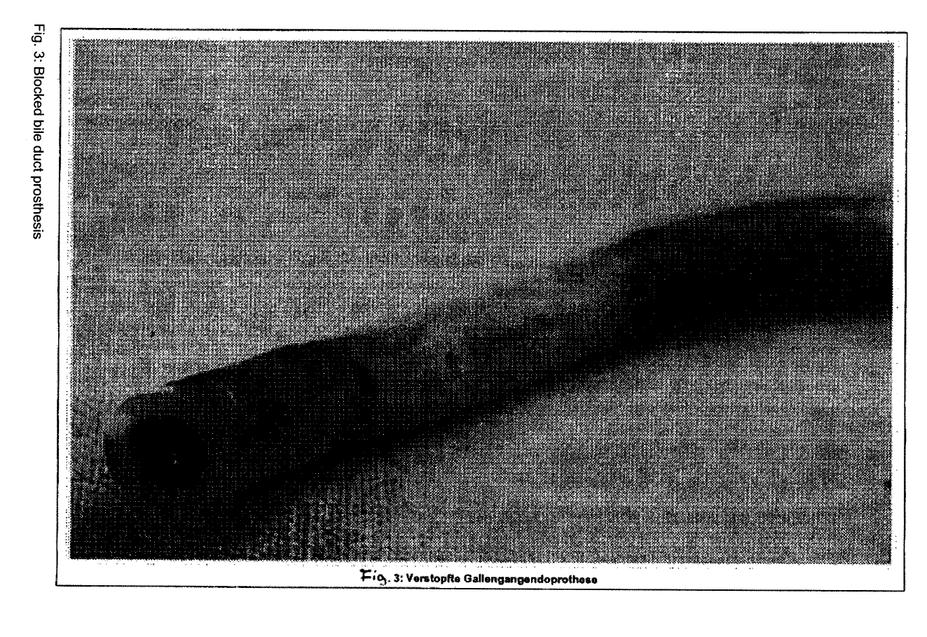

FIG. 3 shows a blocked plastic stent, which would need to be removed due to insufficient drainage.

Up to now, different approaches have been used to solve the blockage problem.

New endoscopes with larger working channels made it possible to place stents with larger diameters. SPEER et al. described prostheses with a diameter of up to 10 French in order to be able to achieve longer lying times. However, even larger diameters could not achieve further improvements in the drainage duration.

Since the bilirubinate produced by the bacterial β glucuronidase is quantitatively a main component of the sludge, an attempt was made in several randomized studies to restrict the sludge formation through prophylactic antibiotic measures.

However, additionally administered ursodeoxycholic acid or acetylsalicylic acid lead clinically to no detectable improvement in the drainage performance.

LEUNG et al. were also unable to detect in-vitro an advantage for the bile-prevalent antibiotics. However, they were able to prove a tendency towards lower sludge deposits in an in-vitro experiment with silver-coated stents. However, this effect did not show an advantage clinically.

Different plastics are used in the production of bile duct prostheses.

The endoscopic department at UKE Hamburg currently uses Teflon.

Based on the lowest friction coefficients of all plastic materials and thus a very smooth surface as well as hydrophobic properties, an advantage was determined both in-vitro and clinically.

Different plastic stents, which were coated with hydrophilic polymers, showed experimental indications of lower sludge formations and deposits. However, this effect could not be substantiated in subsequent in-vivo studies.

Since 1990, the “Christmas tree” prosthesis has been used instead of the “Pigtail” prosthesis originally introduced at the UKE in Hamburg in 1979. It has a straight shape and no side holes. In accordance with FIG. 1, three rows with four ears each are cut into the prosthesis wall for anchoring without perforating them. Due to the fact that side holes were not used, a low blockage rate was determined. This also resulted in a decrease in re-interventions.

Biliary metal stents represent an alternative, which can be obtained commercially as a self-expanding “wall stent” or balloon-expanding “Strecker stent” and as an “endocoil stent.”

Up to now, the most commonly used is the “wall stent.” The maximum prosthesis diameter is 1 cm; the prosthesis system is not normally technically complicated. However, it is problematic that the metal stents are very expensive (approx. 1,500 Euros), without thereby significantly increasing the survival rate compared to plastic prostheses, although the occlusion rate is significantly lower (33% with metal vs. 54% for plastic). The long-term drainage is impaired by tumor growth into the prosthesis.

The further-developed coated metal stents allow a longer drainage period due to their silicon or polyurethane surface.

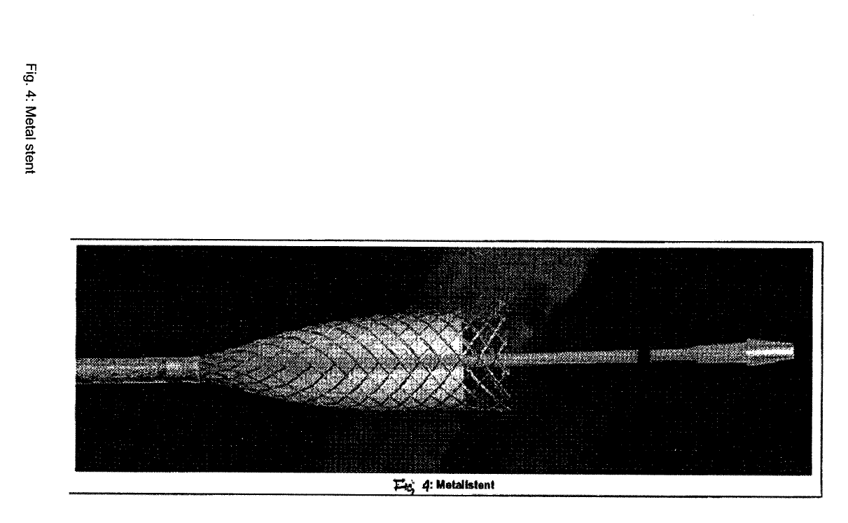

Since a metal stent can no longer be removed, this late complication must be individually coordinated and treated by inserting a plastic prosthesis through the metal stent, a second metal stent, but also using thermal processes. FIG. 4 shows a commercial metal stent.

When budgets are tight, the use of metal endoprostheses is problematic.

For a subgroup of patients in good general condition, with a lack of distant metastases and an estimated survival rate of more than 6 months, its early application is advantageous with respect to the quality of life and cost.

Nanocoatings are also already known for bile duct prostheses. “Nano” is derived from the Greek word for dwarf and describes the encroachment into low dimensions on the nanometer scale (nm), one millionth of a millimeter.

The nanotechnology is concerned with systems, the functions and properties of which are determined by their small spatial structures. Typically, an enlargement is less than 100 nm. Components of the nanotechnology are thus only made up of a few atoms or molecules.

The nanoparticles have clearly changed properties in their behavior with respect to larger solid state bodies, since they have a very large ratio of volume to surface compared to larger particles. Thus, despite the same chemical basis, drastic property changes are possible.

The inorganic/organic coating materials based on sol-gel have proven themselves as a very innovative developmental direction in the field of nanotechnology. These materials are characterized by the extraordinary variation width of their properties, the composition and homogeneity of which can be control in the molecular area. They thereby represent the consequential application of known chemical synthetic principles for the development of new substance materials.

As an example, smooth surfaces can be modified such that dirt or water can no longer adhere to it.

Some of the new materials arising from the sol-gel process are already being used industrially, e.g. as scratch-proof automobile paint and dirt-resistant outdoor paint.

The basic technology behind the synthesis of sol-gel coating materials is described below.

Hydrolyzed inorganic compounds are used as the base material in the technical sol-gel process. Alkoxy compounds of different elements are the most important.

The basic principle is explained in FIG. 5 using the example of an alkoxy compound of the silicon (silicic acetate).

The basic structure of this silicon ester: Si(OR)₄

OR here stands for hydrolysable groups, which are separated as alcohols during the reaction.

Alkoxides of the silicon are generally also called silanes.

In the first reaction step, silane compounds are hydrolyzed. This results in the partial formation of reactive silanols (Si—OH), in which alcohol remains on the silicon are replaced by OH groups. As a general rule, the formed alcohols remain in the sol.

The hydrolysis leads to a reactive intermediate product, the sol, which is made up of colloidal particles. It is a low-viscosity and colorless liquid, which can function as a coating material.

In this stage, the reactive monomer and oligomer pre-stages are created in the colloidal solution for a later cross-linking reaction, which are a few nanometers in size.

In a second reaction step, a condensation reaction, the sol is converted to polysiloxanes (gel state). This either happens at room temperature or upon supply of thermal energy. Inorganic oxidic polymer structures, a filigree network of nanoparticles, are created.

With organically modified components, which are integrated into the sol-gel process, it is now possible to produce materials that are not porous and more flexible than pure inorganic polymers.

In order to combine the inorganic and organic polymers, different organically modified alkoxides can be used, e.g. organosilanes with one or more organic groups instead of alkoxy functions.

The organic groups then anchored on the organic network give the composite material controllable properties.

This results in the following structure: (OR)₃Si—R₁ (R₁=alkyl remainder, functional group)

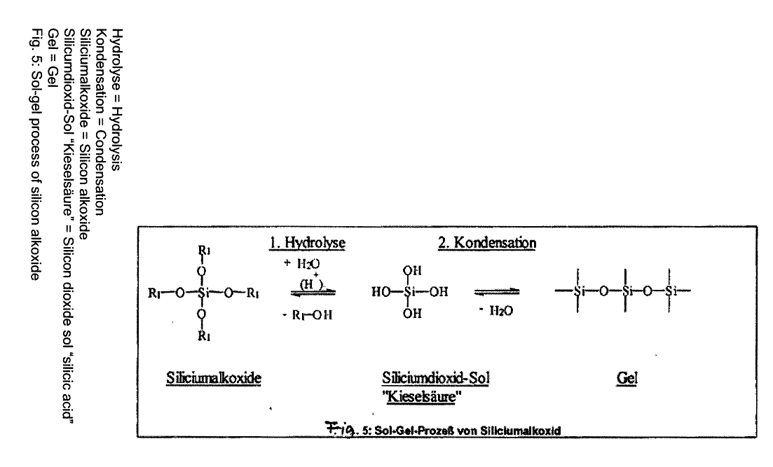

FIG. 6 shows schematically the effect of different organic/inorganic structure elements on the properties of the hybrid materials.

Through hydrophobization of the sol-gel matrix and the resulting formation of a low-energy surface, a dirt- and water-resistant coating e.g. can be reached.

The higher the inorganic portion, the more brittle and hard the resulting layers. The higher the organic portion in the network formation, the higher the flexibility and the density.

Application on surfaces is possible with conventional painting processes such as spraying, dipping, pouring, spinning, etc.

The inorganic/organic hybrid polymers are resistant with respect to most organic solutions. The layers can only be attacked by very strong alkalis, e.g. sodium hydroxide.

Since the production of the aforementioned “classic” hybrid sol-gel materials is still very price-intensive, the Institut für Lack und Farben e.V. Magdeburg has developed an alternative concept for the production of hybrid sol-gel coatings using adhesive agents.

Pure organic polymers, which are characterized by a clearly reduced production price with constant processing properties, are used as organic modification components.

In a two-step process, the organic polymers (adhesive agent) are functionalized using selectively reacting bi-functional coupling substances.

Then the inorganic sol components connect with the still remaining reactive group of the coupling substance on the polymer. A hydrophobization substance is also added.

It was shown through the use of organic polymers in the sol-gel process that a similarly defined and targeted influence of the property image of coating materials can be achieved as through the use of organically substituted silanes.

The object of the present invention is to specify a procedure of the initially named type such that a simple in-vitro experimental setup is provided for examining biofilm formation on endoprostheses.

This object is solved in accordance with the invention such that a coated endoprosthesis is positioned in at least one incubation vessel and that the incubation vessel is at least partially filled with infected bile, as well as in that at least one incubation vessel provided with an endoprosthesis to be examined is slowly tipped and pivoted back and forth, as well as in that a portion of the bile fluid is replaced on a regular basis.

Another object of the present invention is to be able to construct a device of the initially named type such that an inexpensive and better quality in-vitro examination of coated endoprostheses can be performed.

This object is solved according to the invention in that a swiveling table for positioning samples is positioned in a pivotable manner relative to an axis of rotation and in that an incubation vessel at least partially filled with bile is arranged for the incorporation of the samples on the swiveling table.

Another object of the present invention is to construct an endoprosthesis of the initially named type such that improved properties are provided for avoiding or reducing sludge deposits.

This object is solved according to the invention in that the coating has 70 to 80 wt-% hydrophobic components and 30 to 20 wt-% hydrophilic components.

In particular, it has proven to be advantageous that the coating is designed on a sol-gel basis. The endoprosthesis serves in particular to direct fluids within the body. Preferred embodiments are bile duct endoprostheses, bloodstream endoprostheses, urinary tract endoprostheses, intrauterine prostheses as well as at least partially tube-like endoprostheses for the directing of fluids.

A high quality prediction can be achieved in that the bile duct prostheses are swiveled back and forth on the swiveling table within the incubation vessel for approx. 35 days.

A good circulation of the prosthesis with bile with simultaneous minimization of unfavorable movement effects can result from the fact that approx. 10 swivel procedures are performed per minute.

In order to modify the experimental conditions for real application conditions after an implantation of the prosthesis, it is suggested that a temperature of approx. 37° C. be maintained within the incubation vessel.

An extensive convergence of the experimental conditions with real impact parameters takes place in that the sample is designed as a coated bile duct prosthesis.

A less expensive and simultaneously robust and reliable experimental setup is provided in that the swiveling table is coupled with an engine via a drive.

In order to enable the adjustability of the swiveling table, an adjustable engine speed is suggested.

In particular, it is intended that the engine be connected with the swiveling table via a coupling device for establishing a slow back and forth swiveling.

In order to minimize the formation of biofilm, it has proven to be advantageous if the coating contains approx. 75 wt-% hydrophobic components and approx. 25% hydrophilic components.

A preferred material selection consists in that the plastic is made of Teflon. Polyethylene or polyurethane are also possible.

A particularly low biofilm formation can be achieved in that the coating is EP19AEVI.

Through the coating of bile duct prosthesis made of Teflon with hydrophobic inorganic/organic sol-gel materials, not only a water-repelling effect is achieved, but also a reduced adherence for proteins and bacterial biofilm on the smooth surface.

An inexpensive, hydrophobic coating of bile duct stents on a sol-gel basis leads to a lower blockage rate and thus to a lower re-intervention rate for stent replacement.

The stent blockage process can be interrupted by hydrophobized inorganic/organic coating materials. A sufficient palliation of the tumor patient could thus be achieved up until the end of the patient's life.

In order to examine the bile duct prosthesis, an apparatus is prepared that swivels with a low frequency and a maximum tilt angle of 40°, in order to avoid tipping over the fluid incubation medium. The apparatus is also as space- and energy-saving as possible in order to perform the experiment at 37° C. in a bacteria incubator. A slow back and forth swiveling takes place in order to achieve a similar biofilm creation as under in-vivo conditions.

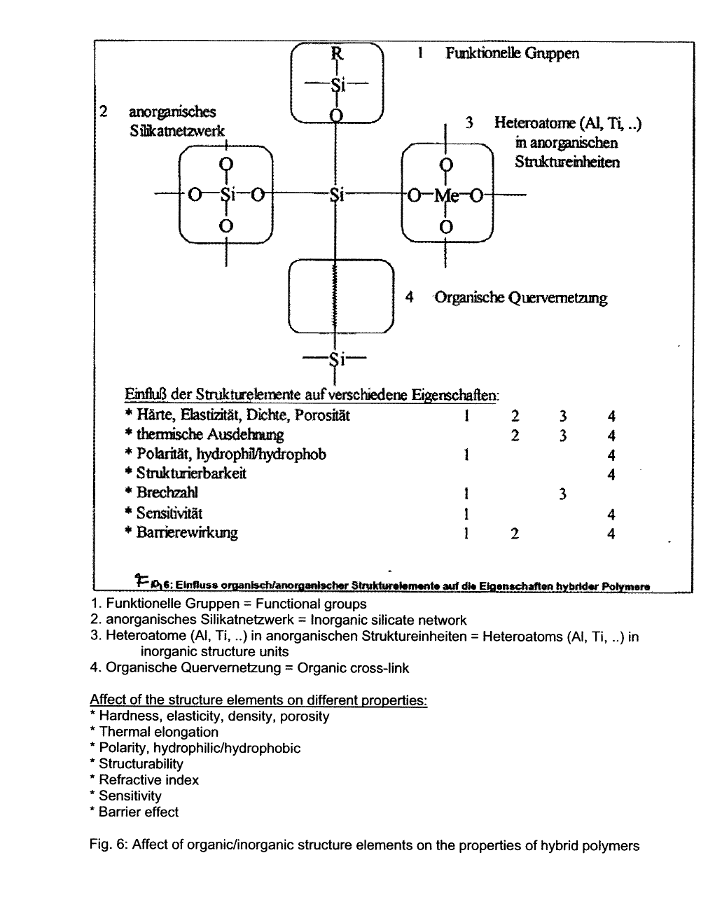

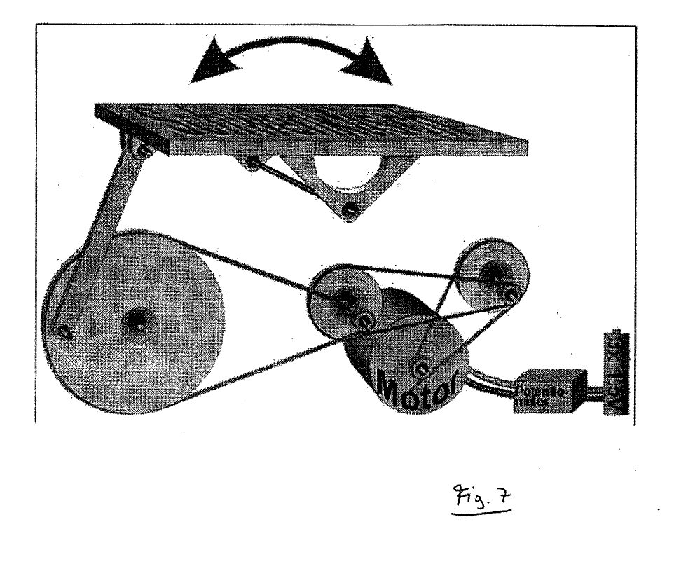

In accordance with the embodiment in FIG. 7 and FIG. 8, a swiveling table with an axis is mounted on a base plate. A cassette recorder engine with a relative high rate of revolution serves as the drive. With the help of three gear wheels as the transmission, this high number is subdivided into roughly 10 revolutions per minute. With a standard potentiometer, which functions as an adjustable voltage divider, the number of revolutions of the table axis can also be regulated precisely. The entire apparatus requires 4.5 volts, which are supplied by three type-C baby cells at 1.5 volts each. The device has a surface area of 18×9 cm and an overall height of 18 cm.

The materials often used for the entire duration of the experiment and their sources are listed in Table 1.

The biological material used to perform the experiment is explained below. In a collective time period of a total of 10 weeks, approx. 1.5 liters of human bile were collected. The collective group was made up of 12 female and 6 male patients between the ages of 42 and 82. All of the patients required interventional endoscopic intervention due to malignant or benign hepatobiliary diseases or need to undergo surgical therapy. Thus, there was no additional effort for the patients for the extraction of the bile.

Each patient was informed of the procedure and the intended use for the bile before the extraction.

It was ensured anamnestically that the patients had not received antibiotic therapy for a period of a week before the extraction.

In the clinic for interdisciplinary endoscopy at the UKE, 4-15 ml of bile was removed from both stationary and ambulant patients via the working channel of the duodenoscope during an ERCP, stored in sterile single-use syringes and deep frozen at −21° C. until further processing.

25-65 ml of bile were extracted from stationary patients of the surgical department of the UKE from a post-operative T-drainage, placed in sterile Falcon tubes and also stored at −21° C.

The main portion of the collected bile comes from stationary patients in the department for endoscopy at the General Hospital in Barmbek (AKB).

A total of approx. 950 ml of bile were extracted from two patients from a post-operative T-drainage. The extraction and storage took place as described above.

The bile from each individual patient was stored and labeled separately on each day of extraction.

Table 2 shows all patients and their diagnoses chronologically by extraction date. The sex, age, extraction type and extraction amount as well as the respective clinic are also listed here.

A bile pool of at least one liter was required for this study.

If possible, the bile should not contain any antibiotics and should be as close to sterile and cell-free as possible.

The collected bile was placed in Falcon tubes in portions of 50 ml for further processing.

Between all processing steps, the bile was always stored at −21° C. in order to prevent an overgrowth of bacteria.

An inhibitor test was performed on each bile sample in order to test for antibiotics.

One drop of bile was placed on a filter plate in an agarose plate inoculated with Bacteroides fragiles (non-spore-forming rod-shaped bacteria, obligate anaerobes, gram-negative) and incubated overnight at 37° C.

If a sample contained an antibiotic, it was detected using the existing inhibitor around the filter paper. An antibiotic was detected for only two patients; their bile was then discarded.

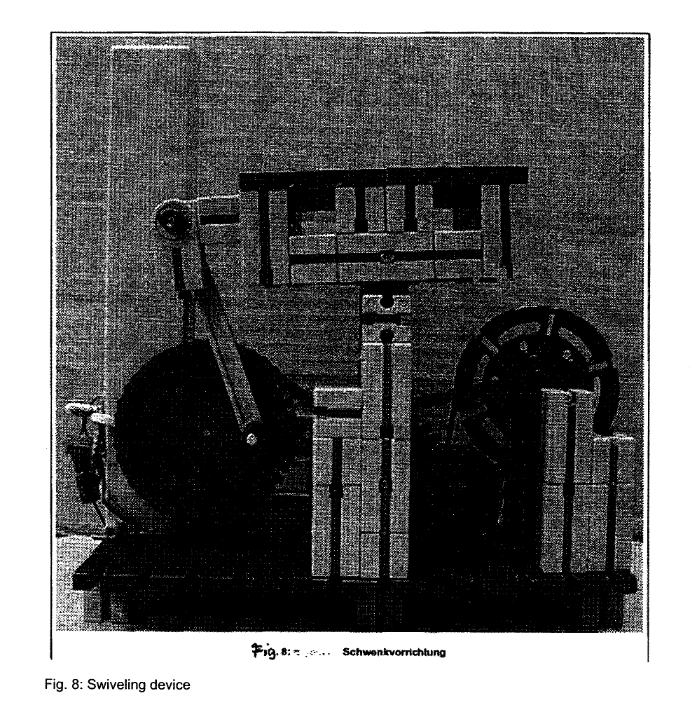

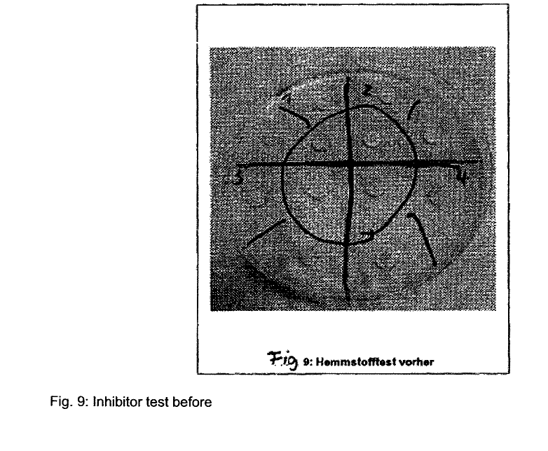

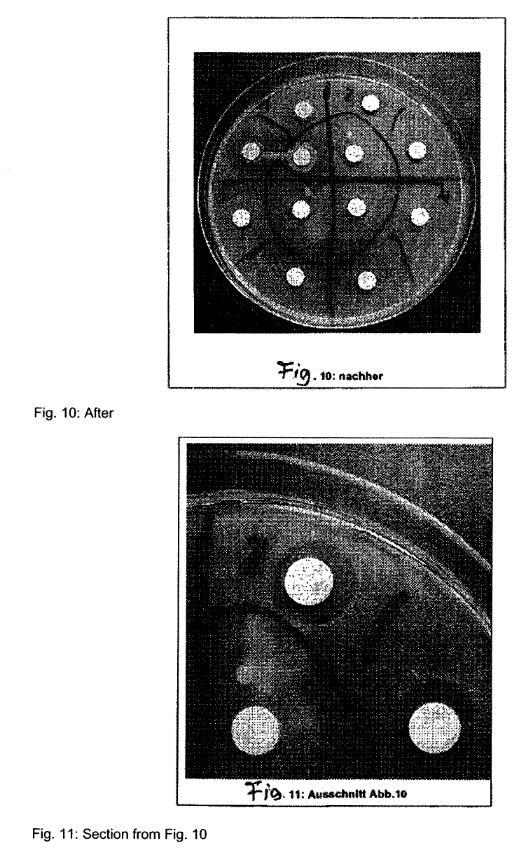

FIGS. 9 and 10 clarify the principle of the inhibitor test.

FIG. 11 clearly shows the inhibitor of an antibiotic-positive sample.

A bile pool was then created out of all other samples.

It was then attempted in a first step to remove through centrifugation all large cells, germs and crystalline precipitates. For this, each sample was centrifuged in a table centrifuge for 20 minutes at 8,000 revolutions per minute.

All of the pellets were discarded. The excess was collected in a glass flask and the poured through a paper filter two time to filter out possible cells, which could not be extracted during centrifugation.

The filtrate was collected in another glass flask and poured into two simple blood bags.

The pool was irradiated for nine minutes in a HWM 400D blood irradiation device made by the company Hans Wällischmiller GmbH under the supervision of Ms. Dr. Lubitz and colleagues at the Institut für Transfusionsmedizin at the UKE.

The irradiation was supposed to ensure the complete sterility of the bile pool.

On a sterile workbench, the sterility was verified through several specimens on agarose plates, each with three days of incubation.

Up until use, the bile pool was again stored in sterile tubes of 50 ml at −21° C.

For the examination of the biofilm creation on bile duct stents, a germ with potential β glucoronidase activity was required.

In addition to the raster-electron-microscopic analyses of the biofilm development on the different coatings, examination should be prepared with a fluorescent microscope.

A standard strain of E. coli (DH5α), which was transformed with a vector from Clontech (pDsRed2; catalogue # 6943-1), fulfilled both of these requirements and was used in all experiments.

The pDsRed2 plasmid contains an origin sequence for the reproduction in bacteria, an ampicillin resistance gene as well as the DsRed2 gene for a red-fluorescent protein (RFP) 2^{nd} generation (further development of the DsRed1) of the Discosoma type.

FIG. 12 shows the plasmid map as well as the Multiple Cloning Site from pDsRed2.

The bacterial β glucoronidase activity was assured in an examination of the Microbiological Institute at the UKE. ID 32 E, an identification system for enterobacteria from the company bioMérieux, served as proof.

FIG. 13 shows the identification of the germ on the reader table. The β glucoronidase activity is marked with a red arrow.

A pure culture of the germ was produced on agar and a pin-head-sized amount was mixed with a commercial phosphate buffer using the tip of a pipette.

This suspension was smoothed out on an object carrier and was examined with a fluorescence microscope to detect fluorescence.

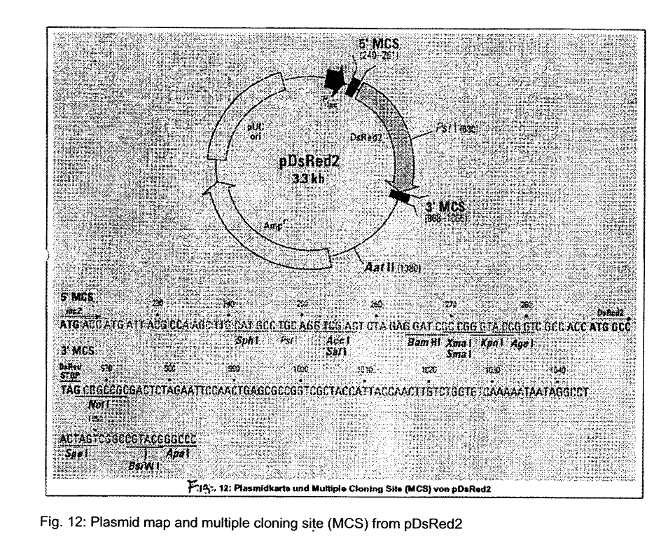

FIG. 14 shows an image of DH5α-pDsRed2 with and without incitation of florescence.

In order to obtain a defined germ amount per milliliter, a bacteria suspension was first produced as follows:

| | |
|---|---|
| LB medium: | 1 g NaCl, 1 g Typton water and 0.5 g yeast |
| | extract were filled to 100 ml with distilled |
| | water and autoclaved for 15 minutes at 121° |
| | C. and 1.5 bar. |
| Ampicillin | 1 g ampicillin power was dissolved in 10 ml |
| solution: | distilled water, the solution was poured |
| | through a sterile filter with 0.25 pore sizes |
| | and was frozen in portions up to 1 ml in |
| | Eppendorf tubes at −21° C. |

100 μm of the ampicillin solution was added to 100 ml of the liquid LB medium.

This created an end concentration of 100 μg ampicillin/ml medium.

A pin-head-sized amount of deep-frozen bacteria was transferred to this liquid culture using the tip of a pipette. The suspension was then incubated in the shaker overnight at 37° C. and 100 upm.

On the next day, 1 ml of the bacteria suspension was mixed with 250 μl of glycerin for the protection of the germs in Eppendorf tubes and stored at −80° C.

From this bacteria suspension, a serial dilution series in logarithmic steps up to 8^{th} potency in order to be able to determine from this the number of germs per milliliter.

| | |
|---|---|
| Agarose | 32 g Lennox L agar from the company Invitrogen |
| plates: | were mixed and autoclaved with 1 l of distilled |
| | water, the still hot, liquid medium was freely |
| | poured into Petri dishes and was allowed to harden. |
| | For ampicillin agarose plates, 1 ml of the ampi- |
| | cillin solution was added to the agarose cooled |
| | to the touch and the mixture was then poured out. |

From each dilution, 50 ml were applied to agarose plates with a glass spatula, which were then incubated overnight at 37° C.

The created colonies of two plates were counted manually and an average germ count of 80×10⁶ germs/ml was determined from the result.

Each frozen tube with 1 ml of the bacteria suspension thus received almost 80 million germs. For the use of bacteria, the tubes can then be individually thawed.

During the experiment, 7 different surface materials and, for comparison, uncoated bile duct stents made of Teflon were examined.

The stent material made of Teflon with a diameter of 12 French were provided by Mr. Hans-Christian Grosse—medical technology/doctor and hospital needs—as well as the raw material made of Teflon for the new coatings.

The new coating concept was developed by the Institut für Lack und Farben e.V. Magdeburg (iLF; Dr. Uwe Wienhold) and applied to ready prostheses made of Teflon.

All products are hydrophobized organic/inorganic coating materials.

The following production method was used:
1.) Functionalization of the adhesive agent (organic polymer) using selectively reacting bi-functional coupling substances.
2.) Conversion of the still remaining reactive group of the coupling substance on the polymer with inorganic sol components and hydrophobizing substance

The coding of the tested materials is described below.

| | |
|---|---|
| Coating 1: | EP19AE/VI-F88/2 |
| Coating 2: | EP50AE/VI-F88/2 |
| Coating 3: | EP19AE/VI-F26/2 |
| Coating 4: | EP50AE/VI-F26/2 |
| Coating 5: | EP19AE/VI |
| Coating 6: | EP50AE/VI |
| Coating 7: | Clearcoat U-111 |

Explanations of the individual coatings:

| | |
|---|---|
| EP19: | is a low-molecular epoxy resin (190 mol) |
| | with a ratio of organic hydrophobic |
| | portions to the inorganic hydrophilic |
| | portions of ~50:50 |
| EP50: | is a high-molecular epoxy resin (500 mol) |
| | with a ratio of organic hydrophobic |
| | portions to the inorganic hydrophilic |
| | portions of ~75:25 |
| AE: | is a coupling substance (amino ethoxy silane) |
| F88 or F26: | are different hydrophobizing substances |
| | (fluorine silane with a coupled amino |
| | group that is hydrophobic) |
| Roman VI: | describes the approach number of the epoxy resin |
| | (e.g. EP19AE/VI is the 6^{th} approach for |
| | the production of a low-molecular epoxy |
| | resin with coupling substance) |
| 2: | is the concentration on hydrophobizing substance |
| Clearcoat | is a hydrophobic commercial sol-gel material |
| U111: | |

Four Teflon stents were coated with each material.

Since the inner coating of the tubes was first only performed as a lab experiment, a complete and optimal coating of the inside could not be guaranteed on the part of the manufacturer.

Nonetheless, all manufactured tubes were supplied to the experiments.

Each coating was examined in triplet with bacteria suspension as well as a negative control with the addition of antibiotics.

As a positive control for bacteria growth on surfaces, a simple silicon tube was also examined with an uneven surface.

First, all preparations were halved lengthwise with a scalpel and were cut into approx. 2.5-cm-long pieces.

Then the prosthesis halves were inserted into 12-hole cell culture plates in the horizontal position using tweezers. The inside of the prosthesis were thereby arranged parallel to the swivel direction in order to imitate the physiological bile flow through a stent. So that gravity-caused deposits could be excluded, the samples were also tipped by 90°.

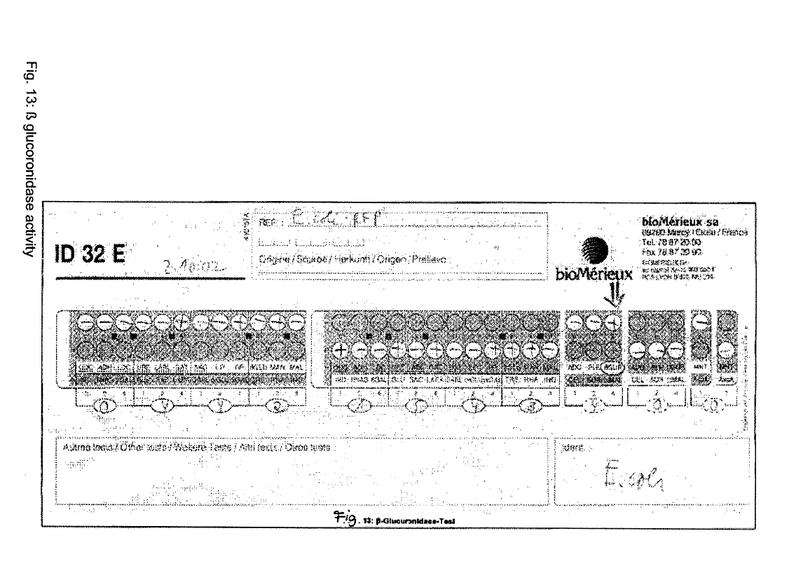

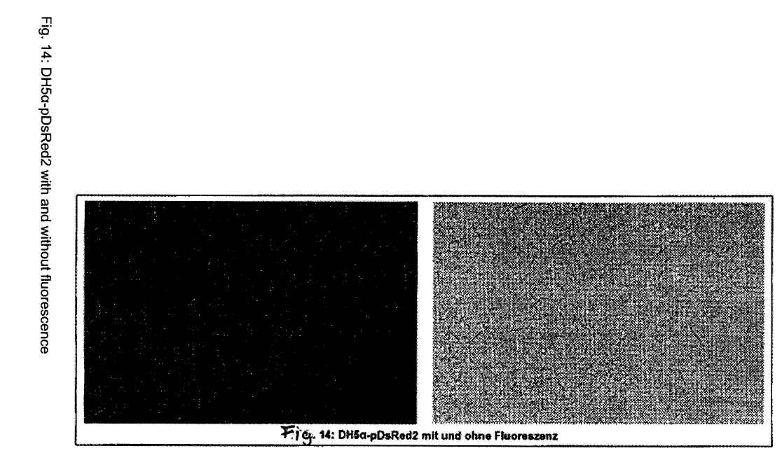

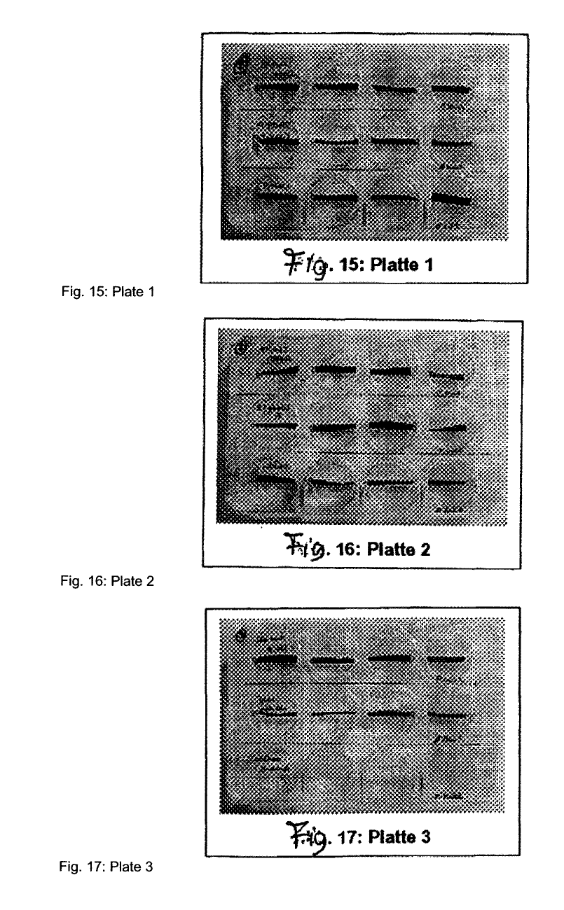

FIG. 15 through 17 show the three printed experimental plates.

The bile pool frozen in the Falcon tubes was thawed in portions in a water bath at 37° C.

The deep-frozen bacteria tubes were slowly heated to room temperature.

Doxycycline “Vibravenös”®SE (Pfizer) was used as the antibiotic.

3 ml of the thawed bile pool were added to each sample in the cell culture plates with a pipette.

Approach with Bacteria:

A tube with 1 ml of a bacteria suspension received 80×10⁶ germs.

In order to receive a defined number of 2×10⁶ germs/ml of bile, 75 μl of suspension were added to three of four samples of each material to be tested.

Approach with an Antibiotic:

The fourth sample of each material was incubated with just one antibiotic without the addition of bacteria as the negative control for the exclusion of bacterial growth.

The antibiotic was also to avoid any contamination by bacteria in the ambient air. The broad-spectrum antibiotic doxycycline provides suitable range of effectiveness.

In order to achieve the working concentration of 10 μm of antibiotic/ml of bile recommended by the local microbiological institute, 1.5 μl of doxycycline were added to the 3 ml of bile.

Table 3 shows the incubation attempts.

The cell culture plates were closed with a cover after filling. The individual plates were also covered with parafilm in order to establish an oxygen-poor milieu suitable for E. coli and to avoid contamination of both the incubator and the swiveling device.

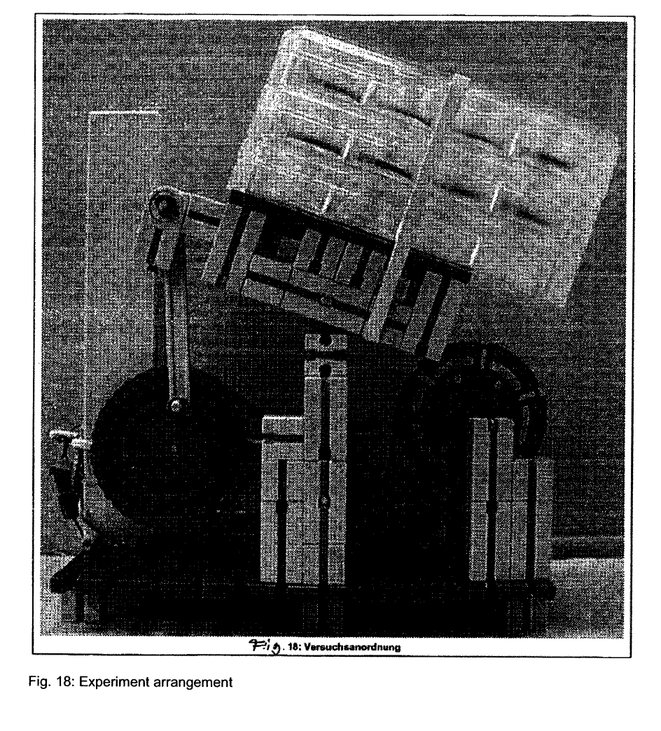

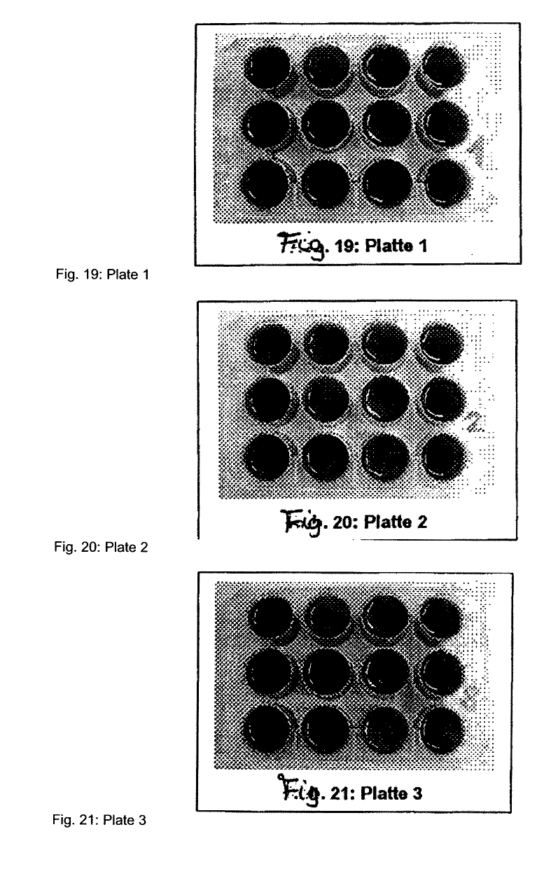

FIG. 18 through 21 show the arrangement of the experiment.

The preparations were incubated for a total of 35 days at 37° C.

The bacteria growth was controlled with daily smearing on agarose plates.

Additionally, the survival of the sensitive germs was examined randomly using a fluorescence microscope every two days throughout the entire period of observation.

In order to avoid a lack of nutrients, 1 ml of bile was removed from the samples with the bacteria suspension every two days and was replaced with 1 ml of fresh bile from the pool. Once per week, the entire bacteria sample was replaced as described above for incubation approach, since a sure survival of the sensitive germs was not guaranteed.

The sterility of the samples with doxycycline was also checked daily through smears. In accordance with a half life of approx. 20 hours, 1.5 μl of doxycycline were re-added to the samples with the antibiotic every 4 days.

The batteries of the swiveling device were replaced weekly to exclude voltage fluctuations and an associated reduction in the swivel frequency.

The correct experimental process was checked twice daily, once in the morning and once in the evening.

In the 35-day incubation period, the samples were examined every two days using a fluorescence microscope in order to detect a potential early die off of the bacteria. The preparation for this and the process of the examination are explained below.

For the examinations under a scanning electron microscope, all samples were transferred to new cell culture plates using tweezers and each of them were set in 3 ml of 3% glutaraldehyde (GA) in 0.05 molar phosphate buffer (PBS). To this end, 12.5 ml of 25% GA and 25 ml of 0.2 m PBS were filled to 100 ml with distilled water. The plates were stored at 4° C. until further preparation.

An Axiovert 135 reflective fluorescence microscope from Zeiss was used to examine the fluorescence of the bacteria. The samples were able to be examined at different levels of magnification. Noticeable findings were captured using an integrated digital camera from Canon.

The fluorescence microscopy procedure is based on the fact that certain molecules give off a portion of the light absorbed by them in the form of longer wave (more energy poor) radiation.

A mercury vapor lamp emits short-wave radiation (white light).

The wave length suitable for the stimulation of the fluorochrom is filtered out in an exciter filter.

A blocking filter, which is only penetrable for long-wave, i.e. “secondary radiation” (fluorescence) created by emissions on the preparation, is located in the between the lens and the eyepiece.

According to the manufacturer, the excitation optimum of DsRed2 lies at 558 nm, and its emission maximum at 583 nm.

Accordingly, suitable excitation and emissions filters were used to observe the samples.

No special preparation was required for the examination of the samples.

The individual samples were removed from the plates filled with bile using tweezers and were observed under the microscope on an object carrier.

Work needed to be performed rapidly in order to prevent the samples from drying out and the dying of the bacteria preparation. After the completion of the examination, the samples were transferred to the cell culture plates for further incubation.

A DSM 940 electron microscope from Zeiss (Germany) was used for this study.

A reflex camera, which could be used to record noticeable findings on the examined samples, was connected with the microscope.

The scanning electron microscopy is suitable for viewing of conductive surfaces. Biological objects must thus first be made conductive by evaporating a metal film.

The usable magnification area ranges from approx. 5 to 100,000 times. At a value of a few nanometers, the achievable resolution capability is approx. 100 times better than that of a light microscope.

By heating a wolfram cylinder (cathode), a primary electron beam is created that focuses through a control cylinder (Wehnelt cylinder) and is accelerated through an anode.

Subsequently, the primary electron beam passes by electromagnetic coils, thereby experiences a fine bundling and hits the object in a focused manner.

A line pattern is created using an XY deflection system. The object surface is scanned line by line, whereby so-called secondary electrons are created. These are captured by a detector; light flashes are created in a scintillator, which are converted back and strengthened electrically from a photomultiplier. In conclusion, this electrical signal is made visible on a monitor.

Other detection methods for capturing infrared radiation electrons or X-rays should be mentioned briefly.

The density and the material composition of an object can hereby be determined.

The object to be observed is immobilized on a sample table in the sample chamber and can be moved in different directions mechanically.
1.) X axis: left and right movement
2.) Y axis: forward and backwards movement
3.) Z axis: up and down movement
4.) Tipping: 0°-90° tipping possible
5.) Rotation: Rotation around its own axis

The basic prerequisite for the examination of organic objects using the SEM are absolutely dry preparations, as well as conductive surfaces.

When drying an organic sample in free air, structure artifacts can be created through strong water loss and the resulting high surface tension.

In order to prevent this, the samples were set in glutaraldehyde and are subsequently dehydrated by means of an increasing alcohol series.

The critical point drying was applied to dry the preparation.

This procedure is based on the fact that there is no difference between the liquid and gaseous aggregate state above the critical point of a liquid and that no effect from voltage on the preparation are to be expected during drying. Thus, the sensitive bacteria and proteins are gently conserved on the prostheses.

At 374° C. and 220 bar, the critical point of water is hard to reach. For this reason, carbon dioxide was used since the critical point of 31° C. and 74 bar is easy to reach without destroying the samples.

The samples were stored in an evacuated exsiccator until further processing.

In the case of insulating preparations, the bombardment of electrons leads to supercharges and thus to image distortions. For this reason, the surface is coated with a conductive layer. This is achieved through sputtering.

The SEM coating system from the company Bio-Rad (Microscience Division) was used to apply a gold layer to the samples.

The dried preparations were adhered to pin sample plates previously imprinted with conductive silver in order to enable improved electrical conductivity.

Three preparations were then simultaneously added to the sputter coater.

The air within the chamber was then exchanged with argon so that the electron flow is not diverted by other molecules. A pressure of 0.08 torr is created through rinsing with argon. All preparations were sputtered for approx. 2 minutes with gold. Then the system was re-supplied with air and the preparations coated with gold were removed from the device.

The samples in turn were stored in an exsiccator filled with drying agents and evacuated with a water jet pump until observation in an electron microscope.

The 35 samples of the experiment as well as an unused sample of each material were first looked over randomly with the SEM in order to get an overview of typical or noticeable findings. More exact examinations of all surfaces took place with up to 10,000 times magnification. At least three images were captured of each sample as documentation.

Additionally, the samples were divided into different groups in order to be able to better detect and describe potential commonalities and differences.

The coatings with low and high molecular epoxide shares were compared both among themselves and against each other.

The effect of the hydrophobic substances was also compared with each other.

The following examination groups were selected at random for the later evaluation:

| | | |
|---|---|---|
| Group 1: | Low-molecular epoxy resin | EP19AE/VI-F88/2 |
| | (EP19) 190 mol | EP19AE/VI-F26/2 |
| | | EP19AE/VI |
| Group 2: | High-molecular epoxy resin | EP50AE/VI-F88/2 |
| | (EP50) 500 mol | EP50AE/VI-F26/2 |
| | | EP50AE/VI |
| Group 3: | Hydrophobic substance 88 | EP19AE/VI-F88/2 |
| | | EP50AE/VI-F88/2 |
| Group 4: | Hydrophobic substance 26 | EP19AE/VI-F26/2 |
| | | EP50AE/VI-F26/2 |
| Group 5: | Clearcoat U-111 | Clearcoat U-111 |
| | | vs. all other groups |
| Group 6: | Teflon uncoated | Teflon uncoated |
| | | vs. all other groups |
| Group 7: | Silicon tube | Silicon tube |
| | | vs. all other groups |

A continuous perfusion of the examination materials with bile was ensured with the simplified method of the slow back and forth swiveling of the bile in small incubation vessels and the biofilm and sludge formation on the different plastic surfaces was easy to examine.

After a 35-day incubation period, the bile was removed from all cell culture plates with a water jet pump and the samples were examined macroscopically with the naked eye.

The supernatant of the filtered bile pool still showed the presence of germs in 4 of 10 specimens. Since the experiment required germ-free bile, the pool was sterilized with gamma radiation.

After the irradiation with 30 Gy in a blood irradiation device from the Institute for Transfusion Medicine at the UKE, all specimens were germ-free and the bile could then be considered sterile.

Most samples had a firmly adhering mucus film. The prostheses were partially encrusted.

However, the mucus on the surfaces of the stents with the coatings EP50AE/VI and EP19AE/VI was not as firmly fixed and dissolved in the fixation medium during the fixation for the scanning electron microscope.

It was also observed that the mucus on the uncoated Teflon stents was thicker and more solid than on all other samples.

The silicon tube became so porous that it almost crumbled when touched with the tweezers. Nonetheless, these samples were processed in the same manner as the others, but they were not included in the evaluation.

Approach with Bacteria

The number of germs in the incubation method was determined through regular smears on agarose plates. The created colonies were counted manually; germ counts of 1.9×10⁶ through 3.7×10⁶/ml of bile were detected. The targeted number of at least 2×10⁶ germs per ml of bile was able to be maintained with the regular additional of free germs.

Approach with an Antibiotic

The samples without the addition of bacteria proved to be completely germ-free for the first two weeks of the incubation period. After this, bacterial contamination was identified in individual specimens, although there was a considerably smaller germ count of approx. 1.5×10⁶ germs/ml of bile compared to the samples with the addition of bacteria. However, through a one-time increase in the regularly added dosage of doxycycline to 20 μg (corresponds to 3 μl of antibiotic/ml of bile), the germs in these specimens were able to be completed killed off so that all further smears were free of germs.

Fluorescence microscopic examinations aided in the observation of the progress of the experiment.

Thus, in addition to the smears on agarose plates, the survival of the bacteria could be checked every two days.

Based on the expression of the fluorescence plasmid pDsRed2, the germs lit up bright red on the surfaces of the stent. However, it was not possible to differentiate between firmly adhering and free bacteria.

Since no ampicillin was added to the incubation medium, the germs lost their fluorescence plasmid under this selection pressure; the expression of the red fluorescence could thus only be observed for a period of approx. 24 to a maximum of 36 hours. However, in the light microscope without fluorescence excitation, living germs were still clearly visible. Through the regular addition of fresh bacteria suspension, the fluorescence was able to be observed throughout the entire time period.

Below is a description of the noticeable findings; all samples of the different coatings were thereby combined.

Although the smears of the respective 4^{th} sample of an examination series were germ-free after interim contamination, bacteria were also found on these samples using a scanning electron microscope. However, the number was not comparable with the germ counts on the other prostheses, so that only the findings of the samples with the addition of bacteria were described below. These samples also showed no protein film or sludge formation.

These results will first be presented individually. Then we will compare the formed examination groups.

The raw state of the unused samples shows the fine channel structure of the surface of the prostheses. The surfaces of the coated stents were thereby contoured more sharply than that of conventional Teflon stents. However, the fine nanostructuring of the individual coatings could not be illustrated with the resolution capability of the REM, so that no differences could be detected between the surfaces. The silicon tube also had a finely channeled surface, which seemed very washed-out and almost mucus-like.

### A 1-4: EP19AE/VI-F88/2:

Thick coatings were detected. Bacteria were immured into large conglomerates in several layers. The bacteria were partially connected via strands, which were similar to appendages and thus also established connections to crystalloid structures. More than half of the overall surface was covered with sludge, the single-layered protein film was distributed evenly like a film over the entire surface. Diffuse individual bacteria were also distributed. The channeled stent surface was detectable in all preparations. But some places showed irregularities and more bacteria adhered there.

### B 1-4: EP50AE/VI-F88/2:

In these preparations, the bacteria conglomerates mainly showed up on the upper edge of the channels. The bacteria were partially interconnected. The multi-layered sludge progressed in bands over the prostheses. The surface could no longer be identified. Overall, less than half of the prostheses were covered with sludge. A thin, even film layer could be detected, which appeared to be ripped at two places and detached itself from the underlayer. The fine channels of the stent surfaces could still be detected in the spots without sludge. Individual bacteria were also distributed all over the entire surface.

### C 1-4: EP19AE/VI-F26/2:

Thick conglomerates with multi-layered, immured bacteria that were also interconnected via their pili were seen. There were also structures that were similar to plant fibers and that were embedded in the even protein film layer. Free surfaces were partially detected, which were covered with a thin film, but not with bacteria. Overall, more than half of the surface was covered. At some spots, the protein film was detached from the surface and the sludge lying over it was ripped. Many diffusely distributed bacteria, partially grown into the film, could be seen over the entire surface.

### D 1-4: EP50AE/VI-F2612:

The film layer appeared to be net-like and uneven. It was chipped in some places. Above all, sludge conglomerations formed on these uneven surfaces. They covered less than half of prostheses, but had many layers. The surface structure was partially no longer detectable. Individual bacteria were distributed everywhere, some of which sometimes formed short chains and had a strand-like connection with the film layer. Crystalloid structures were immured in the protein film and formed contact surfaces for bacteria clusters.

### E 1-4: EP19AE/VI:

A regular, thin film layer appeared over the entire surface of these prostheses. Only individual bacteria clusters could be detected, which were also only partially multi-layered. Overall, less than 25% of the prostheses surfaces were coated with sludge. The main portion formed individual, diffusely distributed bacteria, which did not form any conglomerates. Large areas of the prostheses were entirely free of bacteria and those areas only had a very thin protein film.

### F 1-4: EP50AE/VI:

There was no sludge on these samples. There was a very thin film over the entire surface. Overall, the structure of the surface was very clearly visible. In addition to large bacteria-free areas, there were only loosely distributed, individually lying bacteria. One sample showed a defect in the surface that looked like a deep groove. This was the only spot where the bacteria laid more closely together and formed interconnections. These bacteria clusters were similar to the sludge observed on the prostheses with other coatings, but were considerably thinner.

### G 1-4: Clearcoat U-111:

A thick protein film, in which many bacteria were partially immured in multiple layers, was seen over the entire surface. The channeled surface structure could still be detected. There were, in particular, thicker bacteria layers on the protein film, but overall less than half of the surface was covered with sludge. The film was ripped in several spots. It was very easy to see the enormous thickness of the film here.

### H 1-4: Teflon Uncoated:

Multi-layered sludge was seen on a generalized, thick protein film.

Free spots could no longer be detected on all samples. Bacteria adhered everywhere over the entire surface. The channel structure of the prostheses was still very difficult to see. At some spots in one sample, the film layer was ripped and the stent surface appeared. The thickness of the protein film and the multiple layers of the bacteria clusters were easy to see.

The samples were already very porous before the fixation for the scanning electron microscopy. The sputtering only worked in two of the four samples.

Swollen material surfaces could then be detected. The bacteria seemed to eat into the material. Overall, it was difficult to identify structures with the SEM.

These samples were then removed from the evaluation.

Table 4 summarized the evaluation of the sample findings.

Table 4 is used as the basis to compare the coatings. For an explanation of the fields, see the description of the legend for Table 4 in Chapter 4.6.3.

Group 1: Low-molecular epoxy resin (EP19) 190 mol

| | Sludge | Protein Film | Individual Bacteria |
|---|---|---|---|
| 1 EP19AE/VI-F88/2 | +++ | ++ | ++ |
| 3 EP19AE/VI-F26/2 | +++ | ++ | ++ |
| 5 EP19AE/VI | + | ++ | + |

Only isolated bacteria clusters were seen on the prostheses with the coating EP19AE/VI in contrast to the prostheses with added hydrophobizing substance (EP 19AE/VI-F88/2 and EP19AE/VI-F26/2). Many more bacteria deposits were detected here and more than half of the surface was covered with sludge. The film on the prostheses with EP19AE/VI coating was also much thinner. The EP19AE/VI-F88/2 coating in turn showed a more even film layer than in the prostheses coated with EP19AE/VI-F26/2, which often have ripped or accumulated protein layers. In the field of low-molecular epoxy resins, the EP19AE/VI coating without additional hydrophobization has proven to be advantageous compared to the hydrophobized substances with respect to the sludge formation.

Group 2: High-molecular epoxy resin (EP50) 500 mol

| | Sludge | Protein Film | Individual Bacteria |
|---|---|---|---|
| 2 EP50AE/VI-F88/2 | ++ | ++ | ++ |
| 4 EP50AE/VI-F26/2 | ++ | ++ | ++ |
| 6 EP19AE/VI | − | + | + |

Overall, the high molecular epoxy resin showed the best results. The EP50AE/VI coating stood out. There were no large sludge deposits on these prostheses. Entirely bacteria-free areas could be detected over and over again. The film layer of the hydrophobized coatings (EP50AE/VI-F88/2 and EP50AE/VI-F26/2) was also so thin than for EP50AE/VI, but appeared partially net-like and irregularly and was even ripped in several spots. An increased number of bacteria clusters were then formed on these irregular spots. It remains to be determined that the EP50AE/VI coating did not show any typical sludge development.

Group 3: Hydrophobic substance 88

| | Sludge | Protein Film | Individual Bacteria |
|---|---|---|---|
| 1 EP19AE/VI-F88/2 | +++ | ++ | ++ |
| 2 EP50AE/VI-F88/2 | ++ | ++ | ++ |

The comparison of these two coatings shows an advantage for the high-molecular substance EP50AE/VI-F88/2. Here, less than half of the prostheses surfaces were covered with sludge, while, on the low-molecular coatings EP19AE/VI-F88/2, thick layers were formed on more than 70% of the surface.

Group 4: Hydrophobic substance 26

| | Sludge | Protein Film | Individual Bacteria |
|---|---|---|---|
| 1 EP19AE/VI-F26/2 | +++ | ++ | ++ |
| 2 EP50AE/VI-F26/2 | ++ | ++ | ++ |

In comparison, this hydrophobization substance has a distribution pattern similar to substance 88. The high molecular coating EP50AE/VI-F26/2 again proved to be advantageous with respect to the low-molecular coating.

EP19AE/VI-F26/2. Thick conglomerates with multi-layered bacteria clusters were detected there and extended over more than half of the surface, while less than 50% of the prosthesis was covered with sludge for EP50AE/VI-F26/2.

### Group 5: Clearcoat U-111

Clearcoat U-111 takes up an intermediate position between the high molecular and the low-molecular coatings. This commercial coating did have advantages in the area of sludge formation with respect to the EP19AE/VI-F88/2 and EP19AE/VI-F26/2 coatings, but had a much thicker protein film than all other coatings. Overall, much more individual bacteria were also distributed over the entire surface than for both EP19AE/VI and EP50AR/VI coatings.

### Group 6: Teflon Uncoated

In this group, it was noticeable that none of the samples had bacteria-free areas. There was an area-wide, multi-layered sludge that also appeared more solid macroscopically than on all other coatings. Individual bacteria on a thick film layer also adhered everywhere on the surface. The stent structure was very hard to detect and appeared washed out. In some cases, it was no longer detectable. In comparison with the new coating materials, the conventional Teflon material did the poorest with respect to the problem of this work due to the clearly stronger sludge formation.

### Group 7: Silicon Tube

The silicon tube was removed from the evaluation, since the material began to dissolve towards the end of the incubation period and could also not be prepared for the scanning electron microscopy in the same manner as the other samples. The viewing with the SEM was only possible with one sample. Thus, the appendix only includes one image of a silicon piece in the raw state as well as one image of the prepared tube.

A comparison with the other coatings could not be performed.

The following list of coating materials and their ability to prevent the formation of sludge (sequence from 1.)=well suited to 8.)=poorly suited) results from the comparison of the randomly formed examination groups as well as from the evaluation of the individual coatings:

1.) EP50AE/VI (high molecular epoxy resin without hydrophobization substance)
2.) EP19AE/VI (low molecular epoxy resin without hydrophobization substance)
3.) EP50AE/VI-F88/2 (high molecular epoxy resin with hydrophobization substance 88)
4.) EP50AE/VI-F26/2 (high molecular epoxy resin with hydrophobization substance 26)
5.) Clearcoat U-111 (commercial hydrophobic coating material)
6.) EP19AE/VI-F88/2 (low molecular epoxy resin with hydrophobization substance 88)
7.) EP19AE/VI-F26/2 (low molecular epoxy resin with hydrophobization substance 26)
8.) Teflon uncoated (conventionally used stent material)

The hydrophilic nature of the materials is of particular importance. This is because cells never bond directly with a material, but first attach proteins, on which the cell adhesion takes place. The protein attachment, which first enables the adsorption of cells, thus depends considerably on the surface properties of the material. Hydrophilic materials promote and hydrophobic materials inhibit the cell attachment.

The described sol-gel coatings are not completely smooth, but have in contrast a surface structured in the nanometer range. However, it has such as small diameter that the contact angle between the surface and the moistening liquid is reduced to a minimum and the adhesive strength is reduced. Round drops of liquid can then roll off these surfaces and simultaneously prevent the adhesion of dirt particles or bacteria. Drops of liquid slide on entirely unstructured smooth surface, such as Teflon. However, only little dirt removal takes place.

All surfaces, regardless of their structure and their chemical composition, can be divided into two groups with respect to the adhesion of water. They are either hydrophilic or hydrophobic. Aqueous liquid are distributed evenly on hydrophilic surfaces, while they roll off of hydrophobic surfaces. A similar classification can be performed for oily materials, e.g. dirt particles.

Human bile is made up of sodium, calcium, chloride, bicarbonate, bile salts, cholesterol, phospholipids, bilirubin and proteins, and is thus a heterogeneous mixture that contains both aqueous as well as oily parts.

Thus, in general: surfaces of bile duct prostheses should be hydrophobic and oleophobic in order to prevent an encrusting with sludge.

However, examinations and practical application showed that even the up to 100% hydrophobic Teflon is not protected from blockage by sludge.

The sol-gel coatings examined here, which have both hydrophobic and hydrophilic components, have significantly reduced sludge formation compared to conventional Teflon material, wherein the best effect was observed for a high molecular coating with a hydrophobic/hydrophilic ratio of 75:25 (EP50AE/VI). A stronger hydrophilization in turn also showed a stronger sludge decrease (all low molecular coatings with a hydrophobic/hydrophilic ratio of 50:50).

TABLE 1
Devices used, chemicals and consumables
Devices:

| | | |
|---|---|---|
| Centrifuge | Type: 5804R | Eppendorf |
| | Rotor: F 34-6-36 (6 × 85 ml) | |
| Water bath | Thermonix BU | Braun |
| Freezer −21° C. | comfort | Liebherr |
| Freezer −80° C. | | Kryotec |
| Incubator | Type: 2771 | Kötterman |
| Autoclave | Type: 3005 | GFL |
| Sputter device | Sanoclav | Wolf |
| Electron microscope | SEM coating system | Bio-Rad |

Chemicals:

| | | |
|---|---|---|
| NaCl | | J. T. Baker |
| Trypton water | #1.10859.500 | Merck |
| Yeast extract | #1.03753.500 | Merck |
| LB agar (Lennox L agar) | #22700-025 | Invitrogen |
| Glutaraldehyde solution | #1.04239.1000 | Merck |
| Ampicillin powder | #835269 | Roche |
| Glycerin | #1.04093.1000 | Merck |

Consumables:

| Batteries | | Procell 1.5 V Size C | Duracell |
|---|---|---|---|
| Pipette tips | 1000 blue | #740296 | Greiner |
| | 200 yellow | #739296 | |
| Cell culture plates | | #150628 | Nunc |
| (12-well-plate) | | | |
| Cell culture plates | | #662102 | Greiner |
| (24-well-plate) | | | |
| Petri dishes | | #663102 | Greiner |
| 50 ml - Falcon tubes | | #2070 | |
| 1.5 ml - Tubes | | #616201 | Bacton Dickinson |
| Fold filter 150 mm | | #10312245 | Schleicher & Schuell |
| Blood bag | 500 ml | #P4162 | Fresenius |
| Compoflex ® | 150 ml | #P4158 | Hemo Care |

TABLE 2
Patients and Diagnoses

| | | | | Extraction | | | | |
|---|---|---|---|---|---|---|---|---|
| Pat. | Sex | Age | Diagnosis | type | Amount | | Date | Clinic |
| 1 | F | 84 | Carcinoma of the pancreas | T-drainage | 25 | ml | Jul. 25, 2002 | UKE |
| | | | head | | | | | |
| 2 | F | 66 | Carcinoma of the pancreas | T-drainage | 50 | ml | Jul. 25, 2002 | UKE |
| | | | head | | | | | |
| 3 | F | 84 | Choledocholithiasis, | ERCP | 15 | ml | Jul. 27, 2002 | UKE |
| | | | cholangitis, pancreatitis | | | | | |
| 4 | M | 42 | Duodenumerh. | T-drainage | 65 | ml | Aug. 20, 2002 | UKE |
| | | | pancreas head res. | | | | | |
| | | | with chronic pancreatitis | | | | | |
| 5 | F | 77 | Choledocholithiasis with BII | ERCP | 6 | ml | Aug. 20, 2002 | UKE |
| | | | stomach and biliary | | | | | |
| | | | pancreatitis | | | | | |
| 6 | F | 75 | Sicker bleeding after | ERCP | 6 | ml | Aug. 26, 2002 | UKE |
| | | | papillotomy with pancreatitis | | | | | |
| | | | and stones | | | | | |
| 7 | F | 66 | Cholestasis with | ERCP | 5 | ml | Aug. 26, 2002 | UKE |
| | | | choledocholithiasis | | | | | |
| 8 | M | 72 | Stent replacement with | ERCP | 10 | ml | Aug. 27, 2002 | UKE |
| | | | Klatskin tumor | | | | | |
| 9 | M | 67 | Stent replacement | ERCP | 5 | ml | Sep. 09, 2002 | UKE |
| 10 | F | 50 | Choledocholithiasis | ERCP | 6 | ml | Sep. 10, 2002 | UKE |
| 11 | M | 66 | Adenoma of the pancreatic | ERCP | 10 | ml | Sep. 09, 2002 | UKE |
| | | | duct, chron. rec. pancreatitis | | | | | |
| 12 | F | 52 | Bile duct stricture | ERCP | 5 | ml | Sep. 16, 2002 | UKE |
| 13 | F | 77 | Choledocholithiasis | ERCP | 5 | ml | Sep. 19, 2002 | UKE |
| 14 | F | 82 | Carcinoma of the gall | ERCP | 5 | ml | Sep. 20, 2002 | UKE |
| | | | bladder | | | | | |
| 15 | F | 62 | Bile colic with | ERCP | 4 | ml | Sep. 24, 2002 | UKE |
| | | | choledocholithiasis | | | | | |
| 16 | F | 67 | Cholestasis | ERCP | 15 | ml | Sep. 26, 2002 | UKE |
| 17 | M | 51 | Biliary peritonitis with | T-drainage | 500 | ml | Oct. 09, 2002 | AKB |
| | | | gastroenterostomy due to | | | | | |
| | | | carcinoma of the pancreas | | | | | |
| | | | head | | | | | |
| 18 | M | 52 | Cholecystectomy due to | T-drainage | 150 | ml | Oct. 10, 2002 | AKB |
| | | | cholecystolithiasis with PSC | | 350 | ml | Oct. 11, 2002 | |

AKB = Allgemeines Krankenhaus Barmbek, Hamburg (Barmbek General Hospital, Hamburg)
UKE = Universitätsklinikum Eppendorf, Hamburg (Eppendorf University Clinic, Hamburg)

TABLE 3
Incubation Preparations of the Cell Culture Plates

| | | | 1 | 2 | 3 | 4 |
|---|---|---|---|---|---|---|
| Plate 1 | A | EP 19 AE/VI-F88/2 | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | B | EP 50 AE/VI-F88/2 | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | C | EP 19 AE/VI-F26/2 | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| Plate 2 | D | EP 50 AE/VI-F26/2 | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | E | EP 19 AE/VI | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | F | EP 50 AE/VI | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| Plate 3 | G | Clearcoat U-111 | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | H | Teflon uncoated | 3 ml bile + | 3 ml bile + | 3 ml bile + | 3 ml bile + |
| | | | 75 μl bact. sus. | 75 μl bact. sus. | 75 μl bact. sus. | 1.5 μl doxy |
| | I | Silicon tube | 3 ml bile + | 3 ml bile + | 3 ml bile + | 3 ml bile + |
| | | | 75 μl bact. sus. | 75 μl bact. sus. | 75 μl bact. sus. | 1.5 μl doxy |

TABLE 4
Surface Properties

| | | Sludge | Protein Film | Individual Bacteria |
|---|---|---|---|---|
| 1 | EP 19 AE/VI-F88/2 | +++ | ++ | ++ |
| 2 | EP 50 AE/VI-F88/2 | ++ | ++ | ++ |
| 3 | EP 19 AE/VI-F26/2 | +++ | ++ | ++ |
| 4 | EP 50 AE/VI-F26/2 | ++ | ++ | ++ |
| 5 | EP 19 AE/VI | + | ++ | ++ |
| 6 | EP 50 AE/VI | — | + | + |
| 7 | Clearcoat U-111 | ++ | +++ | ++ |
| 8 | Teflon uncoated | +++ | +++ | ++ |
| 9 | Silicon tube | 0 | 0 | 0 |

Legend for Table 4:

| | | |
|---|---|---|
| Sludge | Protein Film | Individual Bacteria |
| +++ multiple layers, | +++ multiple layers, even | ++ even, diffuse |
| >50% of surface | | |
| ++ multiple layers, | ++ one layer, even | + isolated |
| 25-50% of surface | | |
| + multiple layers, | + very thin, even | 0 cannot be assessed |
| <25% of surface | | |
| — not available | 0 cannot be assessed | |
| 0 cannot be assessed | | |

1. Method for examining sludge deposits on materials for the coating of endoprostheses, characterized in that at least one coated endoprosthesis is positioned in at least one incubation container and in that the incubation container is at least partially filled with infected bile and in that at least one incubation container provided with an endoprosthesis to be examined is tilted back and forth and in that part of the bile fluid is replaced on a regular basis.

2. Method in accordance with claim 1, characterized in that sludge deposits on bile duct prostheses are examined.

3. Method in accordance with claim 2, characterized in that approx. 10 swivel processes are performed per minute.

4. Method in accordance with claims 1 through 3, characterized in that the swivel process is performed with a tilt angle of approx. +/−20 degree relative to the horizontal plane.

5. Method in accordance with one of claims 1 through 4, characterized in that approx. 1 ml of bile fluid is replaced approx. every 2 days.

6. Device for examining materials for the coating of endoprostheses, characterized in that a swiveling table for the positioning of samples is arranged in a pivotable manner relative to an axis of rotation and in that at least one incubation container filled at least partially with bile is arranged on the swiveling table for the acceptance of samples.

7. Device in accordance with claim 6, characterized in that the sample is a coated bile duct prosthesis.

8. Device in accordance with claim 6 or 7, characterized in that the swiveling table is coupled with an engine via a drive.

9. Device in accordance with one of claims 6 through 8, characterized in that the engine speed can be adjusted.

10. Device in accordance with one of claims 6 through 9, characterized in that the engine is connected with the swiveling table via a coupling device for establishing a slow back and forth swiveling.

11. Endoprosthesis made of a plastic with a coating, characterized in that the coating has 70 to 80 wt-% hydrophobic components and 30 to 20 wt-% hydrophilic components.

12. Endoprosthesis made of a plastic with a coating, characterized in that the coating is designed based on a sol-gel process.

13. Endoprosthesis in accordance with claim 11 or 12, characterized in that approx. 75 wt-% hydrophobic components and approx. 25 wt-% hydrophilic components are contained in the coating.

14. Endoprosthesis in accordance with one of claims 11 through 13, characterized in that the plastic is made of Teflon.

15. Endoprosthesis in accordance with one of claims 11 through 14, characterized in that the coating is EP50AEVI.

16. Endoprosthesis in accordance with one of claims 11 through 14, characterized in that the coating is EP19AEVI.

17. Endoprosthesis in accordance with one of claims 11 through 16, characterized in that the surface of the coating is nanostructured.

18. Endoprosthesis in accordance with one of claims 11 through 17, characterized in that the coating has a thickness of a few nanometers.

19. Endoprosthesis in accordance with one of claims 11 through 18, characterized in that the coating has both organic and inorganic components.

20. Endoprosthesis in accordance with one of claims 11 through 10, characterized in that the coating can be connected to the plastic via an adhesive agent as the coupling substance.

21. Endoprosthesis in accordance with claim 20, characterized in that the coupling substance is aminoetoxsilan.

22. Endoprosthesis in accordance with one of claims 11 through 21, characterized in that the coating has a scratch-proof surface.

23. Endoprosthesis in accordance with one of claims 11 through 21, characterized in that the coating has an elastic surface.

24. Endoprosthesis in accordance with one of claims 11 through 23, characterized in that there is an embodiment as a bile duct prosthesis.

25. Endoprosthesis in accordance with one of claims 11 through 23, characterized in that there is an embodiment as a prosthesis for use in a bloodstream.

26. Endoprosthesis in accordance with one of claims 11 through 23, characterized in that there is an embodiment as a prosthesis for use in a urinary passage.

27. Endoprosthesis in accordance with one of claims 11 through 23, characterized in that there is an embodiment as an intrauterine prosthesis.

28. Endoprosthesis in accordance with one of claims 11 through 23, characterized in that there is an embodiment that is tubular in at least certain areas for guiding fluids.
